(19) United States
(12) Patent Application Publication
(10) Pub. No.: US 2016/0045134 A1
(43) Pub. Date: Feb. 18, 2016

(54) ECG MONITOR WITH AN IMPLANTABLE PART

(71) Applicant: WIDEX A/S, Lynge (DK)

(72) Inventors: Rasmus Stig JENSEN, Lyngby (DK); Soren Erik WESTERMANN, Humlebak (DK); Richard TOPHOLM, Holte (DK)

(73) Assignee: WIDEX A/S, Lynge (DK)

(21) Appl. No.: 14/875,794

(22) Filed: Oct. 6, 2015

Related U.S. Application Data
(63) Continuation-in-part of application No. PCT/EP2013/057800, filed on Apr. 15, 2013.

(51) Int. Cl. A61B 5/042; A61B 5/0432; A61B 5/00

(52) U.S. Cl. CPC A61B 5/0422; A61B 5/0006; A61B 5/0022; A61B 5/0432; A61B 2560/0219

(57) ABSTRACT

An ECG monitor comprising an electrode part 1 adapted for subcutaneous implantation and an external part 2 to be carried by a person implanted with the electrode part. The electrode part is provided with at least two electrode areas 6 for detecting an ECG signal when in use, and said electrode part 1 is connected to said external part 2 through a wireless link when in use, said wireless link being adapted for transferring data representing said detected ECG signal to said external part and being adapted for transferring power from said external part to said electrode part.

### RELATED APPLICATIONS

The present application is a continuation-in-part of application PCT/ EP2013057800, filed on 15 Apr. 2013, in Europe, and published as WO 2014169940 A1.

### BACKGROUND OF THE INVENTION

1. Field of the Invention

The present invention relates to ECG monitors. The invention, in particular, relates to EEG monitors of the types which can be continuously carried by a person being monitored. The invention further relates to a personal, wearable ECG monitor comprising an implantable electrode part with at least two electrode areas for measuring an ECG signal of a person, said electrode part comprising an electronic circuit arranged in a housing with the electrode areas arranged externally or on the external side of the housing.

ECG is the commonly used abbreviation for Electro Cardio-Graphy, which is the electrical activity of the heart measured over a period of time by electrodes arranged on the skin surface. In the following the term ECG will also be used for the detection of the electrical activity of the heart by subcutaneously arranged electrodes.

It is known to measure ECG by placing electrodes on the skin surface at different locations of the chest, arms and feet. This type of measurements has been known for a long time, and is used for obtaining momentary ECG measurement or for surveillance during a period of hospitalization.

For some purposes, it is necessary to monitor ECG signals of a person continuously for a longer period of time, e.g. several months or even years. This may be for research purposes, where it might be beneficial to log the ECG signal for later analysis and perhaps correlation with other parameters.

For such purposes, dependence on skin electrodes and their reliability in having sufficient contact to the skin is difficult. Also, irritation of the skin surface could be the result of long term fastening of electrodes by tape or patch. Removing and replacing the electrodes may also introduce some uncertainty about the exact positioning.

2. The Prior Art

Systems for measuring ECG signals in implanted devices are known from US 2007/0179388 A1, which describes a device for subcutaneous measurement of ECG, and recording this in a loop memory. If triggered the device may save a short period of ECG recording permanently.

U.S. Pat. No. 8,241,221 B2 discloses an ECG monitor for subcutaneous implantation and set up for stroke detection. This monitor can be in wireless contact with an external unit, e.g. arranged at the bedside, for alarm giving purposes.

US 2011/0224520 A1 discloses a subcutaneous ECG monitor where the implant has wireless access to an external unit, and can store a parameter indicative of a property or an evaluation result. Access to the monitor is enabled via the internet for visualizing individual patient values or a condition to a physician.

In ECG monitors with implanted electrodes there will also be implanted electronics for sampling the ECG signal, and either transmitting the signal to a non implanted part, or processing the signal in the implanted part. This requires a power source in the implant, and also a reliable wireless connection.

One problem in the known subcutaneously implantable ECG monitors is that the power source is a traditional battery. This takes up space, leading to a larger implant, and is expensive since an implanted battery has to be much safer in relation to leakage of any compounds than standard batteries. An implanted battery also has to be very reliable due to the trouble involved in changing the battery.

Also, the wireless links described in the known implantable ECG monitors would not be suitable for continuous transfer of data, since this would take up too much power, but rather for the transfer of short notifications, such as an alarm, or maybe for transfer of a short period of data, such as a few minutes, to obtain an instant picture of the ECG signal. Another problem of the prior art is that long term storage of ECG signals is not possible.

### SUMMARY OF THE INVENTION

The invention, in a first aspect, provides an ECG monitor comprising an electrode part adapted for subcutaneous implantation and an external part to be carried by a person implanted with the electrode part, where said electrode part is provided with at least two electrode areas for detecting an ECG signal when in use, and said electrode part is connected to said external part through a wireless link when in use, said wireless link being adapted for transferring data representing said detected ECG signal to said external part and being adapted for transferring power from said external part to said electrode part.

A major advantage of this ECG monitor is that it can operate without a battery in the implantable electrode part, or maybe with just a small rechargeable battery with capacity for in the order of 20 minutes operation. A capacity of 20 minutes should be sufficient for the person to have a bath, or to change battery in the external part. In this case a smaller memory capacity in the implantable electrode part sufficient for these 20 minutes of data should also be preferred.

In an embodiment of the ECG monitor, the wireless link is based on an inductive coupling between an internal coil in the electrode part and an external coil in the external part. This provides an efficient wireless system for transfer of both data and power for the electronic in the electrode part.

In an embodiment of the ECG monitor, the external part is provided with an indicator providing notification about the alignment between the coil in the electrode part and the coil in the external part. This ensures that the power transfer is efficient, and that all data are transferred without losing any data.

In an embodiment of the ECG monitor, the external part is provided with means for holding the external part in position at the skin surface at the place where the coil of said electrode part is placed when said electrode part has been implanted. Such means are discussed further below, and should be prepared for easy alignment of the external part.

In an embodiment of the ECG monitor, the data representing said detected ECG signal are continuously logged in the external part. Such data may be the digital representation of the analogue ECG signal. The continuous logging secures a complete ECG data history for the person monitored.

In an embodiment of the ECG monitor, the implantable electrode part does not comprise a battery for chemical storage of energy. This has space, cost and safety benefits.

In an embodiment of the ECG monitor, the external device comprises a link adapted for wireless connection to a remote device, e.g. a smart phone, and for transfer of data to the remote device. This facilitates extra data logging capacity, interface possibility to the monitor, and internet access.

In an embodiment of the ECG monitor, the electrode part is provided with a housing comprising electronics for the wireless link, and the housing is connected to an elongated cable part comprising electrode areas for ECG monitoring. Alternatively or supplementary, electrode areas could also be arranged on the external side of the implantable electrode part housing.

In an embodiment of the ECG monitor, the transfer of ECG data to the external part as well as the transfer of power from said external part to the implantable electrode part are performed simultaneously. This eliminates or reduces the need for memory space and for power storage capability in the implantable electrode part.

In an embodiment of the ECG monitor, the simultaneous transfer of ECG data and power are performed by application of a load modulated power transfer. This has been found to be very reliable and efficient.

The invention, in a second aspect, provides a method for measuring and logging ECG data continuously, said method comprising arranging an external part of an ECG monitor outside the skin opposite the placement of a subcutaneously implanted electrode part, activating said implanted electrode part, transferring power from said external part to said electrode part measuring ECG data by said electrode part and transferring ECG data to said external part, and logging ECG data in said external part.

This method does not necessarily include the step of implanting the electrode part. The method may be performed after the implantation of the electrode part.

In an embodiment of this method the ECG data are logged for at least 90 percent of time per each 24 hour period, for a period of at least one month. This ensures something close to a complete ECG monitoring day after day. Preferably, ECG data are logged for at least 95 percent of time per each 24 hour period. In an alternative embodiment data are logged at least 90 percent of time per each 24 hour period for e.g. 2-5 days each month for several months, e.g. at least 6 months or at least 12 months.

### BRIEF DESCRIPTION OF THE DRAWINGS

Embodiments of the invention will now be explained in further detail with reference to the figures.

FIG. 1 illustrates a possible placement of an ECG monitor at the human body.

FIG. 2 illustrates a cross sectional view of the ECG monitor placement in FIG. 1.

FIG. 3 illustrates a schematic drawing of the two parts of an ECG monitor.

FIG. 4 illustrates the electrode part of the ECG monitor.

FIG. 5 illustrates a side view of the electrode part in FIG. 4.

FIG. 6 illustrates the principle set-up for load modulation.

FIG. 7 illustrates the capacitor coupled in parallel.

FIG. 8 illustrates the capacitors coupled in series.

### DETAILED DESCRIPTION OF THE INVENTION

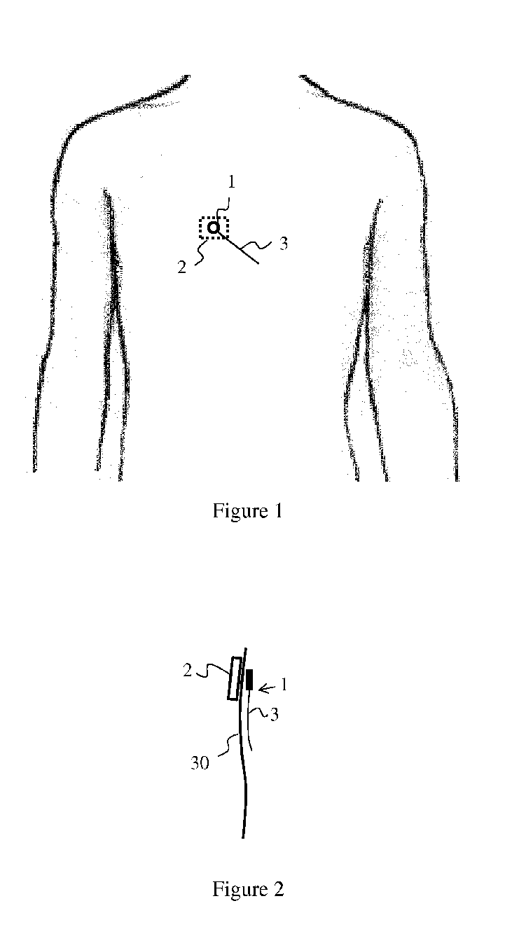

FIG. 1 shows an ECG monitor having an electrode part 1 and an external part 2. It is shown how the implantable electrode part 1 may be arranged in the chest region of the body of the person to be monitored. The electrode part 1 is adapted for being subcutaneously implanted. In FIG. 1 one cable 3 or wire comprising two or more electrode areas is illustrated. But the electrode part 1 may have cables with electrode areas extending in different directions. If the electrode part 1 comprises more than one cable 3, it may be sufficient with one electrode area on one or more cables in order to measure an ECG signal. Electrode areas could also be integrated in the housing of the electrode part. FIG. 1 shows the external part 2 in dotted lines. The external part is in communication with the electrode part 1.

FIG. 2 shows a cross sectional view with a person's skin barrier 30 between an implantable electrode part 1 on the right side and the external part 2 arranged at the skin surface on the left side. It is not shown how the external part is secured to the skin surface on the other side of the implanted part. This securement or attachment may be performed in different ways. One is to attach the external part in a belt or strap which can extend around the body and by resilience hold the external part in the correct position. Another way is to supply both the electrode part 1 and the external part 2 with permanent magnets sufficiently strong to hold the external part in place. The application of patch, plaster or tape is another possibility for attaching the external part.

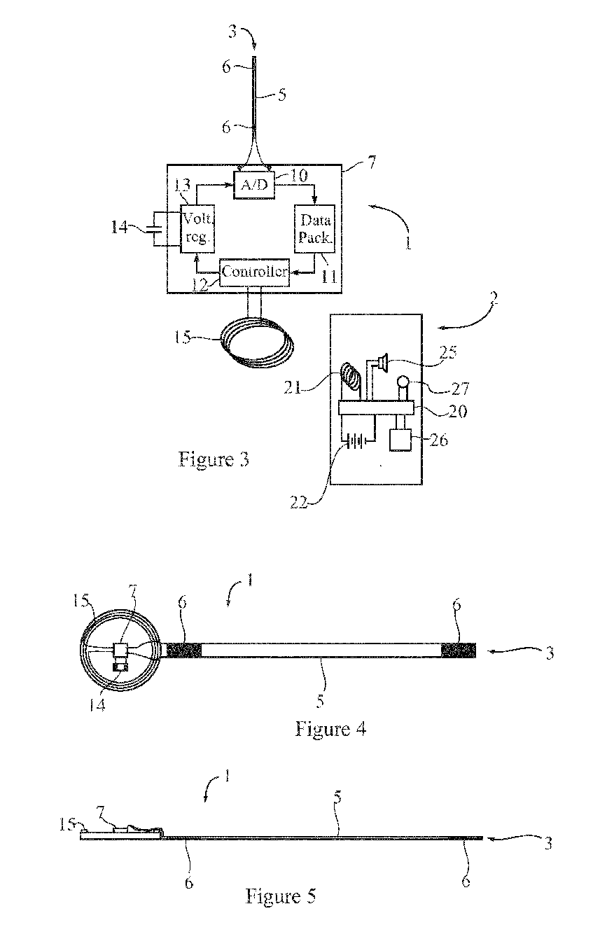

FIG. 3 illustrates an example of an ECG monitoring system comprising an implantable electrode part 1 and an external part 2. The implantable electrode part 1, suitable for being subcutaneously positioned, e.g. in the chest region of a person in need of long term ECG monitoring, comprises a subcutaneous cable 3 having a plurality of active electrode areas 6 separated by insulators 5. The cable 3 is connected to an electronic circuit 7. The cable 3 has at least two electrode areas 6. Often these electrode areas are simply referred to as electrodes. The electronic circuit preferably comprises an A/D converter 10, a data packet controller 12, a communications controller 11, and a voltage regulator 13. The cable with electrode areas is connected to the input terminals of the A/D converter via electrode wires. The communications controller 11, or data packet controller 12, if present, may be connected to an internal coil 15, and the voltage regulator 13, if present, may be connected to a ceramic capacitor 14. As mentioned, other embodiments may have two or more cables 3, each with one or more electrode areas.

The external part 2 of the ECG monitoring system preferably comprises a controller 20 which may be connected to an external coil 21, a battery 22 for powering the controller, and preferably a data storage 26 for saving data representing the measured ECG signal. The external part may comprise a loudspeaker 25 for providing an acoustic signal, e.g. an alarm in the event of a seizure coming up.

When in use, the external part of the ECG monitoring system may be placed at the chest area of a person for whom monitoring of an ECG signal is desired, and in the vicinity of the subcutaneously implantable electrode part 1. The implantable part 1 is often implanted right below the skin at the chest of the person, see FIG. 1, and positioned in such a way that a reliable, electrical ECG signal may be detected by the electrode areas. For this purpose the external part is provided with an indicator 27, which gives some indication if the external part is not arranged or placed such that a sufficient communication between the first and the second communication coils can be achieved.

The electrode areas 6 pick up ECG signals as a varying electrical voltage potential and feed the varying electrical voltage to the input terminals of the A/D converter 10. The A/D converter 10 converts the varying electrical voltage from the electrode areas 6 into a digital signal and may present said digital signal to the data packet controller 11. The data packet controller 11 preferably arranges the digital signal representing the electrical signal from the electrode areas into a stream of data packets according to a predetermined communications protocol, and feeds the resulting stream of data packets to the communications controller 12.

The communications controller 12 is typically configured to perform two operations. The first operation of the communications controller preferably enables the electronic circuit to be energized electromagnetically by receiving energy from the external coil 21 of the external part 2 by the internal coil 15. The electromagnetic energy received in the internal coil 15 may be transferred by the communications controller 12 to the voltage regulator 13 and stored briefly as a voltage charge in the ceramic capacitor 14.

The second operation is the transfer of data where the communications controller may take data packets representing the electrical ECG signals from the electrode areas 6 from the data packet controller 11 and convert them in the internal coil 15 into bursts of electromagnetic energy suitable for being received and detected by the external coil 21 of the external part 2.

If the two operations of the communications controller 12 are performed sequentially, then the electrical energy stored in the ceramic capacitor 14 is used as a power source for the electrical circuit in the implantable electrode part 1, for use during a period where data is transferred.

Data representing the ECG signal may be continuously stored in a data storage 26 or data logger in the external part 2. The ECG data should preferably be logged with time information, and optionally other parameters obtained from other types of sensors, or inputs from the person monitored, may be logged as well. If ECG signal from several pairs of electrode areas are obtained, these should be logged with the same time information. If the external part 2 is in communication with a remote device, data may also be transferred to this device for storage. The remote device could be a mobile phone or any type of portable computer.

The system may also be set up for analyzing the ECG signal continuously for predetermined events. Depending on the results of the analysis of the ECG signals, decisions may be taken by the controller 20 to activate the loudspeaker 25 sounding an alarm, e.g. when a predetermined medical condition is deemed to be present from the analysis of the ECG signals. This alarm may then alert a user to the medical condition, and allow him or her to take adequate steps to alleviate the medical condition, e.g. by taking a prescription drug or consulting medical personnel to ask for immediate advice or help, depending on the medical condition.

An alarm or any other notification from the ECG monitoring system may also be sent to a remote unit. The remote unit may forward the alarm to a hospital or emergency center. An alarm could also be sent directly from the external part to a hospital or emergency center.

FIG. 4 illustrates an example of the implantable electrode part 1 as it could look before being sealed into a housing suitable for implants. Such housing must protect implanted parts against humidity from the body. The housing must also be bio-compatible and protect the person against any harmful compounds leaking from the implant.

The implantable electrode part 1 comprises the cable part 3 with at least two electrode areas 6. The implantable part 1 could comprise more than one cable 3 extending in different directions when implanted. The implantable part 1 further comprises the internal coil 15. This would typically be made relatively large compared to the size of the rest of the implant part 1 in order to facilitate achieving a good coupling to the external coil 21 in the external part 2.

The indicator 27 has the purpose of passing information, e.g. to the person to be monitored, if the coupling coefficient between the two coils 15, 21 is not sufficiently high to facilitate a preselected efficiency of the power transfer, and to obtain a reliable transfer of data. The indicator 27 could have the form of a sound alarm, a visual alarm such as a blinking lamp or a signal sent to another device such as the person's mobile phone or computer.

FIG. 5 illustrates the implantable electrode part of FIG. 4 in a side view. This shows the same components shown in FIG. 4, but illustrates the flatness of the implant. Further to being flat the implant, or at least the cable 3 with the electrode areas 6 of the implantable electrode part, should also be flexible. By having the electrode areas arranged in the wire or cable like part 3, it is possible to achieve a larger distance between electrode areas 6 compared to having the electrode areas 6 arranged directly on the housing for the electronics.

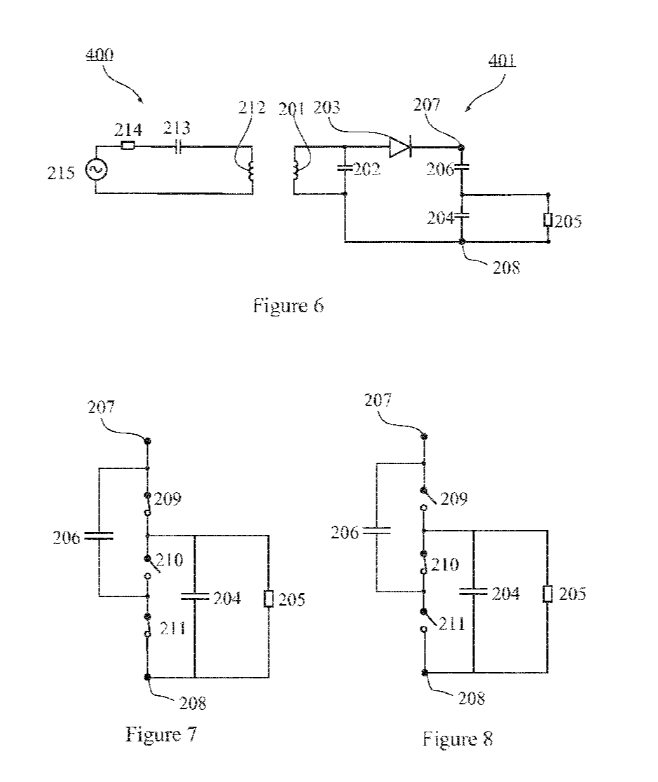

FIG. 6 shows a simplified circuit diagram of an ECG monitor. The ECG monitor has a reader circuit 400 arranged in the external part 2 and a data carrier circuit 401 arranged in the implantable electrode part 1. The reader circuit 400 in the external part includes a signal generator 215, a resonance resistor 214, a resonance capacitor 213 and an external transmitter coil 212. The resonance resistor 214, resonance capacitor 213 and transmitter coil 212 together form a resonant circuit that is tuned to a resonance frequency that corresponds to the chosen transmission frequency of the wireless signal from the external part. The data carrier 401 includes an internal coil 201, a resonance capacitor 202, a rectifier 203, an energy storage capacitor 204, a resistor 205 representing the load of the monitoring and data processing means on the data carrier, a modulation capacitor 206 and connection points 207 and 208.

The signal generator 215 generates an alternating current in the external coil 212. The current in the external coil generates an alternating magnetic field which induces an alternating current in the internal coil 201. The frequency of the alternating magnetic field is denoted the operating frequency. As an example the operating frequency is in the range between 900 kHz and 1100 kHz. The current in the internal coil is used to power the data carrier.

In FIG. 6 the energy storage capacitor 204 and the modulation capacitor 206 are arranged in a serial connection. This will give one impedance when seen from the receiver coil 201, and thereby one specific load on the transmitter coil 212.

Rearranging the energy the storage capacitor 204 and the modulation capacitor 206 into a parallel set-up will change the impedance seen from the internal coil 201, and thereby change the load on the external coil 212.

FIGS. 7 and 8 show a simple way to change between a parallel set-up (FIG. 7) and a serial set-up (FIG. 8) of these two capacitors 204, 206, by simply changing the three switches 209, 210, 211. For simplicity FIGS. 7 and 8 show only the storage capacitor 204, the modulation capacitor 206, the switches 209, 210, 211 and the resistor 205.

In the parallel set-up in FIG. 7 the data carrier forms a low impedance resonant circuit, as seen from a reader inductively linked to the data carrier. In the serial set-up in FIG. 8 the data carrier forms a high impedance resonant circuit. By switching between the two operating states in time with a data stream to be transmitted from the data carrier and to the reader it becomes possible to reconstruct the data stream in the reader based on an appropriate evaluation procedure in the reader, where the modulated load on the transmitter coil 212 will represent data transmitted from the data carrier in the implant to the reader in the external part. It is an advantage that this load modulation is achieved with only negligible power dissipation in the components used for the load modulation. Another advantage is that the load modulated transmission of data runs simultaneously with the transfer of power from the external part to the internal part. This means that it is not necessary to transfer extra power for storage in the implant or electrode part and for use during periods of data transfer to the external part.

The external and internal coils 15, 21 should be closely aligned on each side of the skin barrier in order to achieve an efficient power transfer. The center axis of one coil should preferably continue through the center axis of the other coil, or the two center axes should be arranged close to each other. This close arrangement is ensured when arranging the external part. The electrode part should be placed such during implantation that it will be easy to align the two coils when arranging the external part.

In case of insufficient alignment between the two coils 15, 21 for obtaining a satisfactory power and data transmission, this can be detected by the controller 20, and a notification should be given.

The inductive coupling can be based on frequencies within a wide range. It could be a frequency around 125 kHz, which is also applied in RFID systems. Much higher frequencies may also be applied. This could be a frequency in the range 0.5-2 MHz. But also frequencies in the range around 10 MHz can be applied. The advantage of a higher frequency is that the antennas can be made smaller. Unfortunately, higher frequency will also lead to an increase in power consumption. These tendencies apply to inductive coupling as well as to traditional radio communication. For an inductive coupling where power is to be transferred, the coils must be in the near field of each other in order to obtain an efficient power transfer. In practice this sets the limit for how small the coils can be.

As an example on antenna size for a 1 MHz wireless connection based on inductive coupling, the coil 15 for the implanted electrode part can be made with an outer diameter in the range 10-18 mm, and with a height in the direction perpendicular to the plane defining the outer circumference of the circular coil of 0.5-1.5 mm, preferably about 1 mm. If the coil has a height of 1 mm the height or thickness of the implantable electrode part will be 2-3 mm. A height, or thickness, of approximately 2-3 mm for an implant arranged subcutaneous at the chest region should be acceptable to most people. The corresponding coil 21 in the external part can have an outer diameter in the range 4-10 mm, and a height in the range 4-8 mm, preferably about 6 mm. Such coil sizes will be sufficient for the power transfer as well as for the ECG signal transfer. But the diameters of the coils may also be made larger in order to make the alignment of the external part less critical.

The external part may communicate with a mobile unit, e.g. phone, computer or tablet, for several purposes. One purpose would be to obtain an interface between the ECG monitor and the person being monitored. This interface can be used for checking functionality of the ECG monitor, e.g. need for adjusting placement of external part if the coupling coefficient between the two coils 15, 21 is not optimal. The person should also be notified about any upcoming replacement or recharging of the battery in the external part.

If the ECG monitor is applied for long term surveillance where other parameters are also recorded, and where some parameters should be registered by the person, this could be done through the mobile unit. This could be the person informing about physical activity, eating, well-being etc. Relevant parameters might also be registered and stored with the dataset by the mobile unit itself. This could be temperature, relative humidity, geographical location, level of physical activity (measured as movement of the mobile unit), etc.

The remote or mobile unit may be applied in the setting up of the ECG monitor, since a user friendly interface can easily be made for a mobile unit. The setting up may involve decisions on sampling rate of the signal to be stored, how often data are transferred, e.g. to the mobile unit, when and how to give alarms on e.g. misalignment of coils, replacement of battery in the external part, etc. In general the implantable electrode part is activated when the external part is placed close to it and the coils are aligned. Then it will receive power from the external part, and it will start measuring ECG signals and send these to the external part.

An important function of the mobile unit may be as storage unit for the monitoring data. Data could be transferred from the external part at regular time intervals. The mobile unit could also upload data to an internet server on a regular basis. Regular back-up of stored data on an internet server could minimize the potential loss of data in larger research projects. In a more patient surveillance related use of the monitor, a physician may also detect long term changes in the ECG signal of a patient, if data are transferred to a hospital computer network.

The communication between the external part and the remote unit should preferably be performed at some low power wireless link, such as low power Bluetooth as described in the Bluetooth Core Specification version 4.0, or similar. It is important to keep the external unit as small as possible not to bother the person being monitored too much. For that purpose it is important to keep the power consumption at a minimum, so that a relatively small battery will be sufficient.

For a person being monitored it may be helpful to be able to remove the external part e.g. during sleeping. This would also relieve any effect the attachment means for the external part might have on the skin. For this purpose the external part may be substituted by a different external part during sleeping, where this different external part is provided with a very large coil placed below the bed or below the sheet during night. This different external part would use more power, but could also be plugged into the AC mains.

We claim:

1. An ECG monitor comprising an electrode part adapted for subcutaneous implantation and an external part to be carried by a person implanted with the electrode part, where said electrode part is provided with at least two electrode areas for detecting an ECG signal when in use, and said electrode part is connected to said external part through a wireless link when in use, said wireless link being adapted for transferring data representing said detected ECG signal to said external part and being adapted for transferring power from said external part to said electrode part.

2. The ECG monitor according to claim 1, wherein said wireless link is based on an inductive coupling between an internal coil in said electrode part and an external coil in said external part.

3. The ECG monitor according to claim 2, wherein said external part is provided with an indicator providing notification about the alignment between the coil in said electrode part and the coil in said external part.

4. The ECG monitor according to claim 1, wherein said external part is provided with means for holding the external part in position at the skin surface at the place where the coil of said electrode part is placed when said electrode part has been implanted.

5. The ECG monitor according to claim 1, wherein data representing said detected ECG signal are continuously logged in said external part.

6. The ECG monitor according to claim 1, wherein said electrode part does not comprise a battery for chemical storage of energy.

7. The ECG monitor according to claim 1, wherein said external device comprises a link adapted for wireless connection to a remote device and for transfer of data to said remote device.

8. The ECG monitor according to claim 1, wherein said electrode part is provided with a housing comprising electronics for said wireless link and said housing is connected to an elongated cable part comprising electrode areas for ECG monitoring.

9. The ECG monitor according to claim 1, wherein said transfer of ECG data to said external part as well as said transfer of power from said external part to said electrode part are performed simultaneously.

10. The ECG monitor according to claim 9, wherein said simultaneous transfer of ECG data and power are performed by application of a load modulated power transfer.

11. The ECG monitor according to claim 1, adapted for logging ECG data for at least 90 percent of time per each 24 hour period, for a period of at least one month.

12. A method for measuring and logging ECG data continuously, said method comprising
arranging an external part of an ECG monitor outside the skin opposite the placement of a subcutaneously implanted electrode part,
activating said implanted electrode part,
transferring power from said external part to said electrode part
measuring ECG data by said electrode part and transferring ECG data to said external part, and
logging ECG data in said external part.

13. The method according to claim 11, wherein said transfer of ECG data to said external part as well as said transfer of power from said external part to said electrode part are performed simultaneously.

14. The method according to claim 13, wherein said simultaneous transfer of ECG data and power are performed by application of a load modulated power transfer.

15. The method according to claim 11, wherein ECG data is logged for at least 90 percent of time per each 24 hour period, for a period of at least one month.
